(19) United States
(12) Patent Application Publication
(10) Pub. No.: US 2008/0206146 A1
(43) Pub. Date: Aug. 28, 2008

(54) Functionalized Magnetic Nanoparticles and Methods of Use Thereof

(76) Inventors: Massoud Akhtari, Pasadena, CA (US); Jerome Engel, Los Angeles, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/817,949

(22) PCT Filed: Mar. 21, 2006
(86) PCT No.: PCT/US2006/002377

§ 371 Date: Nov. 9, 2007

Related U.S. Application Data
(60) Provisional application No. 60/664,046, filed on Mar. 21, 2005.

(51) Int. Cl. A61K 49/18; A61K 9/14; A61K 39/395; A61P 25/28; A61K 9/00; A61K 38/02

(52) U.S. Cl. 424/9.3; 424/489; 424/130.1; 424/484; 514/2

(57) ABSTRACT

The present invention provides functionalized magnetic nanoparticles comprising a functional group, which functionalized magnetic nanoparticles exhibit differential binding to a tissue, indulging brain tissue, bone, and vascular tissues. The present invention further provides compositions, including pharmaceutical compostions, comprising a subject functionalized magnetic nanoparticle. The present invention further provides diagnostic and research methods including use of subject functionized magnetic nanoparticles. The present invention further provides a magnetic resonance imaging (MRI)-visible drug delivery system; drugs using MRI, as well as tissue-specific drug delivery.

### CROSS-REFERENCE

This application claims the benefit of U.S. Provisional Patent Application No. 60/664,046, filed Mar. 21, 2005, which application is incorporated herein by reference in its entirety.

### FIELD OF THE INVENTION

The present invention is in the field of magnetic nanoparticles, and their use in imaging, e.g., magnetic resonance imaging, of tissues.

### BACKGROUND OF THE INVENTION

Nanoparticles are very small particles typically ranging in size from as small as one nanometer to as large as several hundred nanometers in diameter. Their small size allows nanoparticles to be exploited to produce a variety of products such as dyes and pigments; aesthetic or functional coatings; tools for biological discovery, medical imaging, and therapeutics; magnetic recording media; quantum dots; and even uniform and nanosize semiconductors.

Use of magnetic nanoparticles has been proposed for various biomedical purposes, including magnetic resonance imaging, hyperthermic treatment of malignant cells, and drug delivery. A major challenge in imaging is the ability to distinguish between diseased tissues and normal tissue. The present invention addresses this need, and provides related advantages.

### LITERATURE

U.S. Pat. Nos. 6,548,264, 6,767,635; Berry and Curtis (2003) J. Phys. D: Applied Physics 36:R198-R206; Pankhurst et al. (2003) J. Phys. D: Applied Physics 36:R167-R181; Dousset et al. (1999) Am. J. Neuroradiol. 20:223-227; Dunning et al. (2004) J. Neurosci. 24:9799-9810; Dousset et al. (1999) Magnetic Resonance in Medicine 41:329-333; Moghimi et al. (2001) Pharmacol. Rev. 53:283-318.

### SUMMARY OF THE INVENTION

The present invention provides functionalized magnetic nanoparticles comprising a functional group, which functionalized magnetic nanoparticles exhibit differential binding to a tissue, including brain tissue, bone, and vascular tissues. The present invention further provides compositions, including pharmaceutical compositions, comprising a subject functionalized magnetic nanoparticle. The present invention further provides diagnostic and research methods involving use of subject functionalized magnetic nanoparticles. The present invention further provides a magnetic resonance imaging (MRI)-visible drug delivery system; as well as methods of synthesizing same. The MRI-visible drug delivery system has applications in determining the distribution of drugs using MRI, as well as tissue-specific drug delivery.

### BRIEF DESCRIPTION OF THE DRAWINGS

FIG. 1 depicts schematically exemplary embodiments of a subject functionalized magnetic nanoparticle.

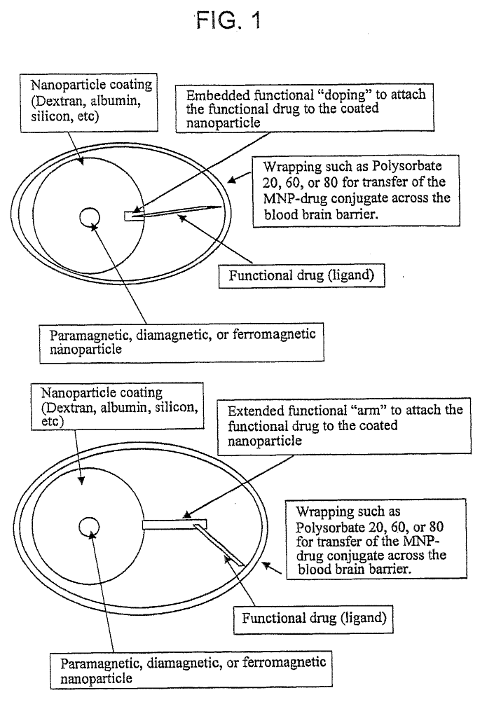

FIGS. 2A-D depict magnetic resonance images of brains of kainic acid-treated rats zero hour after injection with AMT-MNP (FIG. 2A); 6 hours after injection with AMT-MNP (FIG. 2B); zero hour after injection with non-functionalized MNP (FIG. 2C); and 6 hours after injection with non-functionalized MNP (FIG. 2D).

FIGS. 3A-D depict transmission electron microscopy (TEM) images of AMT-MNP particles within a human serum albumin matrix.

FIGS. 4A and 4B depict TEM images of poly(butyl cyanoacrylate)-MNP.

### DEFINITIONS

As used herein, the term “nanoparticle” refers to a particle having a diameter of between about 1 and 1000 nm. Similarly, by the term “nanoparticles” refers to a plurality of particles having an average diameter of between about 1 and 1000 nm.

Reference to the “size” of a nanoparticle is a reference to the length of the largest straight dimension of the nanoparticle. For example, the size of a perfectly spherical nanoparticle is its diameter.

As used herein, the phrase “specifically binds” refers to the situation in which one molecule recognizes and adheres to a particular second molecule in a sample, but does not substantially recognize or adhere to other molecules in the sample. For example, an antibody that “specifically binds” a preselected antigen is one that binds the antigen with a binding affinity greater than about 10⁻⁷ M, e.g., binds with a binding affinity of at least about 10⁻⁷ M, at least about 10⁻⁸ M, or at least about 10⁻⁹ M, or greater than 10⁻⁹ M.

As used herein, the term “functional group,” used interchangeably with “functional moiety” and “functional ligand,” refers to a chemical group that imparts a particular function to an article (e.g., nanoparticle) bearing the chemical group. For example, functional groups can include substances such as antibodies, oligonucleotides, biotin, or streptavidin that are known to bind particular molecules; or small chemical groups such as amines, carboxylates, and the like.

As used herein, “subject” or “individual” or “patient” refers to any subject for whom or which diagnosis, prognosis, or therapy is desired, and generally refers to the recipient of a diagnostic method, a prognostic method, or a therapeutic method, to be practiced according to the invention. The subject can be any vertebrate, but will typically be a mammal. If a mammal, the subject will in many embodiments be a human, but may also be a domestic livestock, laboratory subject, or pet animal.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claimss.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a functionalized magnetic nanoparticle” includes a plurality of such nanoparticles and reference to “the drug” includes reference to one or more drugs and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The present invention provides functionalized magnetic nanoparticles having conjugated thereto a functional moiety, which functionalized magnetic nanoparticles that exhibit differential binding to specific types of tissue, e.g., brain tissue, bone, or vascular tissue. The present invention further provides-compositions comprising subject functionalized magnetic nanoparticles. The present invention further provides diagnostic, prognostic, therapeutic, and research methods involving use of subject functionalized magnetic nanoparticles. The present invention further provides a magnetic resonance imaging (MRI)-visible drug delivery system; as well as methods of synthesizing same. The MRI-visible drug delivery system has applications in determining the distribution of drugs using MRI, as well as tissue-specific drug delivery.

### Functionalized Magnetic Nanoparticles

The present invention provides functionalized magnetic nanoparticles (MNP) having conjugated thereto a functional moiety, which fictionalized magnetic nanoparticles that exhibit differential binding to specific types of tissue, e.g., brain, bone, or vascular tissue. A subject functionalized magnetic nanoparticle is useful for a variety of diagnostic, prognostic, therapeutic, and research applications.

### Magnetic Nanoparticles

Subject nanoparticles generally have a mean size in a range of from about 1 nm to about 1000 nm, e.g., from about 1 nm to about 10 nm, from about 10 nm to about 50 nm, from about 50 nm to about 100 nm, from about 100 nm to about 250 nm, from about 250 nm to about 500 nm, from about 500 nm to about 750 nm, or from about 750 nm to about 1000 nm. Average diameters will in some embodiments range from about 10 nm to about 1000 nm, e.g., from about 10 nm to about 20 nm, from about 20 n to about 40 nm, from about 40 nm to about 60 nm, from about 60 nm to about 80 nm, from about 80 nm to about 100 nm, from about 100 nm to about 200 nm, from about 200 nm to about 400 nm, from about 400 nm to about 600 nm, from about 600 nm to about 800 nm, or from about 800 nm to about 1000 nm.

Nanoparticles can be simple aggregations of molecules or they can be structured into two or more layers of different substances. For example, simple nanoparticles consisting of magnetite or maghemite are suitable for use. See, e.g., Scientific and Clinical Applications of Magnetic Microspheres, U. Hafeli, W. Schutt, J. Teller, and M. Zborowski (eds.) Plenum Press, New York, 1997; and Tiefenauer et al., Bioconjugate Chem. 4:347, 1993. More complex nanoparticles can consist of a core made of one substance and one or more shells made of another substance(s). The term “magnetic nanoparticle” includes paramagnetic nanoparticles, diamagnetic nanoparticles, and ferromagnetic nanoparticles.

Typical core materials of the nanoparticles according to the invention are ferrites of general composition MeOₓFe₂O₃ wherein Me is a bivalent metal such as Co, Mn or Fe. Other suitable materials are γ-Fe₂O₃, the pure metals Co, Fe, Ni, and metal compounds such as carbides and nitrides. The core material is generally an MRI visible agent. The core material is typically coated. Suitable coatings include, but are not limited to, dextran, albumin, starch, silicon, and the like.

Many different type of small particles (nanoparticles or micron-sized particles) are commercially available from several different manufacturers including: Bangs Laboratories (Fishers, Ind.); Promega (Madison, Wis.); Dynal Inc. (Lake Success, N.Y.); Advanced Magnetics Inc. (Surrey, U.K.); CPG Inc. (Lincoln Park, N.J.); Cortex Biochem (San Leandro, Calif.); European institute of Science (Lund, Sweden); Ferrofluidies Corp. (Nashua N.H.); FeRx Inc.; (San Diego, Calif.); Immunicon Corp.; (Huntingdon Valley, Pa.); Magnetically Delivered Therapeutics Inc. (San Diego, Calif.); Miltenyi Biotec GmbH (USA); Microcaps GmbH (Rostock, Germany); PolyMicrospheres Inc. (Indianapolis, Ind.); Scigen Ltd. (Kent, U.K.); Seradyn Inc.; (Indianapolis, Ind.); and Spherotech Inc. (Libertyville, Ill.). Most of these particles are made using conventional techniques, such as grinding and milling, emulsion polymerization, block copolymerization, and microemulsion.

Methods of making silica nanoparticles have also been reported. The processes involve crystallite core aggregation (Philipse et al., Langmuir, 10:92, 1994); fortification of superparamagnetic polymer nanoparticles with intercalated silica (Gruttner, C and J Teller, Journal of Magnetism and Magnetic Materials, 194:8, 1999); and microwave-mediated self-assembly (Correa-Duarte et al., Langmuir, 14:6430, 1998).

Subject nanoparticle cores are magnetic and can include a metal selected from the group consisting of magnetite, maghemite, and greigite. Magnetic nanoparticles can be made using magnetic materials such as magnetite, maghemite, and greigite as part of the core. By varying the overall size and shape of such magnetic cores, they can be made superparamagnetic or stable single-domain (particles that retain a stable magnetic moment after being removed from a magnetic field). Core size relates to whether a magnetic nanoparticle is superparamagnetic or single-domain. Thus, relatively equidimensional superparamagnetic particles generally have a core sized less than 50 to 80 nm. At particle sizes above this upper range, the magnetization of the particle is split into domains of differing magnetization vectors in order to minimize internal magnetic energies.

In some embodiments, the core includes a pigment which can be an inorganic salt such as potassium permanganate, potassium dichromate, nickel sulfate, cobaltchloride, iron(III) chloride, or copper nitrate. Similarly, the core can include a dye such as Ru/Bpy, Eu/Bpy, or the like; or a metal such as Ag and Cd.

In some embodiments, a subject modified nanoparticle comprises a core and a silica shell enveloping the core. The functional group is conjugated to the silica shell, e.g., as described in U.S. Pat. No. 6,548,264. Numerous lnown methods for attaching functional groups to silica can be adapted for use in the present invention. See, e.g., Ralph K. Iler, The Chemistry of Silica: Solubility, Polymerization, Colloid and Surface Properties, and Biochemistry, Wiley-Interscience, NY, 1979; VanDerVoort, P. and Vansant, E. F., Journal of Liquid Chromatography and Related Technologies, 19:2723-2752, 1996; and Immobilized Enzymes. Antigens, Antibodies, and Peptides: Preparation and Characterization, Howard H. Weetall (ed.), M. Dekker, N.Y., 1975. A typical process for adding functional groups to silica-coated nanoparticles involves treating the nanoparticles with a silanizing agent that reacts with and couples a chemical group to the silica surface of the nanoparticles. The chemical group can itself be the functional group, or it can serve as a substrate to which functional groups can be coupled.

For example, in an exemplary method, silica-coated nanoparticles are prepared as described above and the particle surfaces are silanized using trimethylsilylpropyl-diethylenetriamine (DETA), a silanization agent that attaches primary amine groups to silica surfaces. Antibodies or other proteins can then be covalently coupled to the silanized surface using the cyanogen bromide (CNBR) method. As one example, CNBR-mediated coupling can be achieved by suspending silica-coated nanoparticles previously silanized with DETA in a 2 M sodium carbonate buffer and ultrasonicating the mixture to create a particle suspension. A solution of CNBR (e.g., 2 g CNBR/1 ml acetonitirile) is then added to the particle suspension to activate the nanoparticles. After washing the nanoparticles with a neutral buffer (e.g., PBS, pH 8), an antibody solution is added to the activated nanoparticle suspenision causing the antibodies to become bound to the nanoparticles. A glycine solution can also be added to the antibody-coated nanoparticles to block any remaining unreacted sites.

In some embodiments, the magnetic nanoparticle is dextran coated. Magnetic nanoparticles are made using any known process. For example, magnetic iron-dextran particles are prepared by mixing 10 ml of 50% (w/w) aqueous Dextran T-40 (Pharmacia) with an equal volume of an aqueous solution containing 1.51 g FeCl₃-6H₂O and 0.64 g FeCl₂-4H₂O. While stirring, the mixture is titrated to pH 10-11 by the drop-wise addition of 7.5% (v/v) NH₄OH heated to 60-65° C. in a water bath for 15 minutes. Aggregates are then removed by 3 cycles of centrifugation in a low-speed clinical centrifuge at 600×g for 5 minutes. The ferromagnetic iron-dextran particles are separated from unbound dextran by gel filtration chromatography on Sephacryl-300. Five ml of the reaction mixture is then applied to a 2.5×33 cm column and eluted with 0.1 M sodium acetate and 0.15 M NaCl at pH 6.5. The purified ferromagnetic iron-dextran particles collected in the void volume will have a concentration of 7-10 mg/ml as determined by dry weight analysis. Molday and Mackenzie (1982) Journal of Immunological Methods 52:353-367. Also see (Xianqiao (2003) China Particuology Vol. 1, No. 2, 76-79).

In some embodiments, a subject functionalized magnetic nanoparticle is of the formula: M-(L)-Z, the linkage sites between L and Z having covalently bound functional groups, wherein M represents the magnetic core particle, L represents an optional linker group, and Z represents a functional group. In other embodiments, a subject functionalized magnetic nanoparticle is of the formula: M-S-(L)-Z, the linkage sites between S and L and L and Z having covalently bound functional groups, wherein M represents the magnetic core particle, wherein S represents a biocompatible substrate fixed to M, wherein M represents the magnetic core particle, L represents an optional linker group, and Z represents a functional group. Functional groups include moieties that provide for binding to a specific tissue type or cell type; moieties that provide for crossing the BBB; therapeutic agents; and the like.

In some embodiments, a subject functionalized magnetic nanoparticle comprises two or more different functional groups attached to the same core particle. For example, in some embodiments, a subject functionalized magnetic nanoparticle is of the formula M-(L)-Z₁Z₂, or M-S-(L)-Z₁Z₂, where Z₁ and Z₂ are different functional groups. In some embodiments, for example, Z₁ is a tissue-specific binding moiety and Z₂ is a therapeutic agent. In other embodiments, for example, Z₁ is a cell type-specific binding moiety and Z₂ is a therapeutic agent. In other embodiments, for example, Z₁ is a moiety that provides for crossing the BBB; and Z₂ is a therapeutic agent. In other embodiments, for example, Z₁ is a moiety that provides for crossing the BBB; and Z₂ is a tissue-specific binding moiety. In other embodiments, for example, Z₁ is a moiety that provides for binding to a diseased tissue; and Z₂ is a therapeutic agent. In some embodiments, a subject functionalized magnetic nanoparticle comprises at least a third functional moiety Z₃.

The magnetic core particles consist of magnetite, maghemite, ferrites of general formula MeOₓFe₂O₃ wherein Me is a bivalent metal such as cobalt, manganese, iron, or of cobalt, iron, nickel, iron carbide, or iron nitride, as described above. If present, the substrate S is formed by compounds such as polysaccharides or oligosaccharides or derivatives thereof, such as dextran, carboxymethyldextran, starch, dialdehyde starch, chitin, alginate, cellulose, carboxymethylcellulose, proteins or derivatives thereof, such as albumins, peptides, synthetic polymers, such as polyethyleneglycols, polyvinylpyrrolidone, polyethyleneimine, polymethacrylates, bifunctional carboxylic acids and derivatives thereof, such as mercaptosuccinic acid or hydroxycarboxylic acids.

The linker group L is formed by reaction of a compound such as poly- and dicarboxylic acids, polyhydroxycarboxylic acids, diamines, amino acids, peptides, proteins, lipids, lipoproteins, glycoproteins, lectins, oligosaccharides, polysaccharides, oligonucleotides and alkylated derivatives thereof, and nucleic acids (DNA, RNA, PNA) and alkylated derivatives thereof, present either in single-stranded or double-stranded form, which compound includes at least two identical or different functional groups.

A subject functionalized magnetic nanoparticle is capable of passing the blood-brain barrier. For example, a subject functionalized magnetic nanoparticle may comprise, attached to the nanoparticle, or in a formulation with the nanoparticle, or coating the nanoparticle, one or more polymers. Suitable polymers that facilitate crossing of the blood brain barrier include, but are not limited to, surfactants such as polysorbate (e.g., Tween® 20, 40, 60 and 80); poloxamers such as Pluronic® F 68; and the like. In some embodiments, a subject functionalized magnetic nanoparticle is coated with a polysorbate such as, e.g., Tween® 80 (which is Polyoxyethylene-80-sorbitan monooleate), Tween® 40 (which is Polyoxyethylene sorbitan monopalmitate); Tween® 60 (which is Polyoxyethylene sorbitan monostearate); Tween® 20 (which is Polyoxyethylene-20-sorbitan monolaurate); polyoxyethylene 20 sorbitan monopalmitate; polyoxyethylene 20 sorbitan monostearate; polyoxyethylene 20 sorbitan monooleate; etc. Also suitable for use are water soluble polymers, including, e.g.: polyether, for example, polyalkylene oxides such as polyethylene glycol (“PEG”), polyethylene oxide (“PEO”), polyethylene oxide-co-polypropylene oxide (“PPO”), co-polyethylene oxide block or random copolymers, and polyvinyl alcohol (“PVA”); poly(vinyl pyrrolidinone) (“PVP”); poly(amino acids); dextran, and proteins such as albumin. Block co-polymers are suitable for use, e.g., a polyethylene oxide-polypropylene oxide-polyethylene-oxide (PEO-PPO-PEO) triblock co-polymer (e.g., Pluronic® F68); and the like; see, e.g., U.S. Pat. No. 6,923,986. Other methods for crossing the blood brain barrier are discussed in various publications, including, e.g., Chen et al. (2004) Curr. Drug Delivery 1:361-376.

In some embodiments, a subject functionalized MNP comprises one or more agents that provide for evasion of the reticuloendothelial system (RES). Agents that provide for evasion of the RES include, but are not limited to, a block copolymer non-ionic surfactant such as a poloxamine, such as poloxamine 508, poloxamine 908, poloxamine 1508, etc. In some embodiments, a subject functionalized MNP comprises about 1% poloxamine.

Nanoparticles can also be transferred across the blood-brain barrier (BBB) by utilizing the specific delivery channels that are present in the BBB. As one non-limiting example, attachment of alpha-methyl tryptophan to the nanoparticles renders the tryptophan channels receptive to these particles and aids in delivery across the BBB. Other mechanisms are transcytosis and diapedesis, with or without the mediation of the channels present at the BBB.

A subject functionalized magnetic nanoparticle can be delivered to the central nervous system (CNS) using a neurosurgical techniques. In the case of gravely ill patients such as accident victims or those suffering from various forms of dementia, surgical intervention is warranted despite its attendant risks. For instance, a subject functionalized magnetic nanoparticle can be delivered by direct physical introduction into the CNS, such as intraventricular or intrathecal injection. Intraventricular injection may be facilitated by an intraventricular catheter, for example, attached to a reservoir, such as an Ommaya reservoir. Methods of introduction may also be provided by rechargeable or biodegradable devices. Another approach is the disruption of the blood-brain barrier by substances which increase the permeability of the blood-brain barrier. Examples include intra-arterial infusion of poorly diffusible agents such as mannitol, pharmaceuticals which increase cerebrovascular permeability such as etoposide, or vasoactive agents such as leukotrienes. Neuwelt and Rappoport (1984) Fed. Proc. 43:214-219; Baba et al. (1991) J. Cereb. Blood Flow Metab. 11:638-643; and Gennuso et al. (1993) Cancer Invest. 11:638-643.

Further, it may be desirable to administer a subject functionalized magnetic nanoparticle locally to the area in need of diagnosis or treatment; this may be achieved by, for example, local infusion during surgery, by injection, by means of a catheter, or by means of an implant, said implant being of a porous, non-porous, or gelatinous material, including membranes, such as silastic membranes, or fibers.

A subject functionalized magnetic nanoparticle can also be delivered by using pharmacological techniques including chemical modification such that the subject functionalized magnetic nanoparticle will cross the blood-brain barrier. The subject functionalized magnetic nanoparticle may be modified to increase the hydrophobicity of the nanoparticle, decrease net charge or molecular weight of the nanoparticle, or modify the nanoparticle, so that it will resemble one normally transported across the blood-brain barrier. Levin (1980) J. Med. Chem. 23:682-684; Pardridge (1991) in: Peptide Drug Delivery to the Brain; and Kostis et al. (1994) J. Clin. Pharmacol. 34:989-996.

Encapsulation of the subject functionalized magnetic nanoparticle in a hydrophobic environment such as liposomnes is also effective in delivering drugs to the CNS. For example WO 91/04014 describes a liposomal delivery system in which the drug is encapsulated within liposomes to which molecules have been added that are normally transported across the blood-brain barrier.

Another method of formulating a subject functionalized magnetic nanoparticle to pass through the blood-brain barrier is to encapsulate the subject fanctionalized magnetic nanoparticle in a cyclodextrin. Any suitable cyclodextrin which passes through the blood-brain barrier may be employed, including, but not limited to, α-cyclodextrin, β-cyclodextrin and derivatives thereof. See generally, U.S. Pat. Nos. 5,017,566, 5,002,935 and 4,983,586. Such compositions may also include a glycerol derivative as discussed U.S. Pat. No. 5,153,179.

In some embodiments, a subject functionalized magnetic nanoparticle is capable of entering a cell in the brain, e.g., crossing a cell membrane and entering the cytoplasm of the cell. Mechanisms for entering a cell in the brain include, e.g., transcytosis and diapedesis, with or without mediation of appropriate membrane channels.

### Therapeutic Agents

In some embodiments, a subject functionalized magnetic nanoparticle further includes one or more therapeutic agents, for delivery to a tissue, e.g., for targeted delivery to a specific tissue such as a diseased brain tissue, a diseased vascular tissue, or a diseased bone tissue. The nature of the therapeutic agent will depend, in part, on the condition or pathology being treated. For example, where the disorder is epilepsy, suitable therapeutic agents include, but are not limited to, anti-seizure agents. Where the disorder is a brain tumor, suitable therapeutic agents include, but are not limited to, anti-neoplastic agents. Where the disorder is an inflammatory condition of vascular tissue or bone tissue, suitable therapeutic agents include, but are not limited to, anti-inflammatory agents.

Suitable therapeutic agents include, but are not limited to, drugs acting at synaptic and neuroeffector junctional sites; general and local analgesics and anesthetics such as opioid analgesics and antagonists; hypnotics and sedatives; drugs for the treatment of psychiatric disorders such as depression, schizophrenia; anti-epileptics and anticonvulsants; Huntington's disease, aging and Alzheimer's disease; neuroprotective agents (such as excitatory amino acid antagonists and neurotropic factors) and neuroregenerative agents; trophic factors such as brain derived neurotrophic factor, ciliary neurotrophic factor, or nerve growth factor; drugs aimed at the treatment of CNS trauma or stroke; and drugs for the treatment of addiction and drug abuse; autacoids and anti-inflammatory drugs; chemotherapeutic agents for parasitic infections and microbial diseases; immunosuppressive agents and anti-cancer drugs; hormones and hormone antagonists; heavy metals and heavy metal antagonists; antagonists for non-metallic toxic agents; cytostatic agents for the treatment of cancer; radiation therapy immunoactive and immunoreactive agents; and a number of other agents such as transmitters and their respective receptor-agonists and -antagonists, their respective precursors or metabolites; antibiotics, antispasmodics, antihistamines, antinauseants, relaxants, stimulants, “sense” and “anti-sense” oligonucleotides, cerebral dilators, psychotropics, anti-manics, vascular dilators and constrictors, anti-hypertensives, migraine treatments, hypnotics, hyper- or hypo-glycemic agents, mineral or nutritional agents, anti-obesity drugs, anabolics and anti-asthmatics.

A number of suitable therapeutic agents are described in Gilman et al. (1990), “Goodman and Gilman's—The Pharmacological Basis of Therapeutics”, Pergamon Press, New York, and include the following agents:

acetylcholine and synthetic choline esters, naturally occurring cholinomimetic alkaloids and their synthetic congeners, anticholinesterase agents, ganglionic stimulants, atropine, scopolamine and related antimuscarinic drugs, catecholamines and sympathomimetic drugs, such as epinephrine, norepinephrine and dopamine, adrenergic agonists, adrenergic receptor antagonists, transmitters such as GABA, glycine, glutamate, acetylcholine, dopamine, 5-hydroxytryptamine, and histamine, neuroactive peptides; analgesics and anesthetics such as opioid analgesics and antagonists; preanesthetic and anesthetic medications such as benzodiazepines, barbiturates, antihistamines, phenothiazines and butylphenones; opioids; antiemetics; anticholinergic drugs such as atropine, scopolamine or glycopyrrolate; cocaine; chloral derivatives; ethchlorvynol; glutethimide; methyprylon; meprobamate; paraldehyde; disulfuram; morphine, fentanyl and naloxone; centrally active antitussive agents; psychiatric drugs such as phenothiazines, thioxanthenes and other heterocyclic compounds (e.g., halperiodol); tricyclic antidepressants such as desimipramine and imipramine; atypical antidepressants (e.g., fluoxetine and trazodone), monoamine oxidase inhibitors such as isocarboxazid; lithium salts; anxiolytics such as chlordiazepoxyd and diazepam; anti-epileptics including hydantoins, anticonvulsant barbiturates, iminostilbines (such as carbamazepine), succinimides, valproic acid, oxazolidinediones and benzodiazepines; anti-Parldnson drugs such as L-DOPA/CARBIDOPA, D2 and D3 agonists and antagonists, apomorphine, amantadine, ergolines, selegeline, ropinorole, bromocriptine mesylate and anticholinergic agents; antispasticity agents such as baclofen, diazepam and dantrolene; neuroprotective agents, such as excitatory amino acid antagonists, neurotrophic factors and brain derived neurotrophic factor, ciliary neurotrophic factor, or nerve growth factor; neurotrophin(NT) 3 (NT3); NT4 and NT5; gangliosides; neuroregenerative agents; drugs for the treatment of addiction and drug abuse include opioid antagonists and anti-depressants; autocoids and anti-inflammatory drugs such as histamine, bradykinin, kallidin and their respective agonists and antagonists; chemotherapeutic agents for parasitic infections and microbial diseases; anti-cancer drugs including alkylating agents (e.g., nitrosoureas) and antimetabolites; nitrogen mustards, ethylenamines and methylmelamines; allylsulfonates; folic acid analogs; pyrimidine analogs, purine analogs, vinca alkaloids; and antibiotics.

### Functional Moieties

A wide variety of functional groups (moieties) can be attached to a magnetic nanoparticle. Functional groups that are suitable for attaching to a magnetic nanoparticle bind, directly or indirectly, differentially or selectively to a particular, pre-selected brain tissue, a vascular tissue, or bone tissue. As noted above, in some embodiments, a functional group is a therapeutic agent.

By “differential binding” or “selective binding” to a particular tissue (e.g., a brain tissue, a vascular tissue, or bone tissue) is meant that the functionalized magnetic nanoparticle binds to a first tissue in such a manner that the binding to the first brain, vascular, or bone tissue is distinguishable from binding to a second brain, vascular, or bone tissue. For example, in some embodiments, a subject functionalized magnetic nanoparticle binds to a first brain tissue in such a manner that the binding to the first brain tissue is distinguishable from binding to a second brain tissue. In other embodiments, a subject functionalized magnetic nanoparticle binds to a first vascular tissue in such a manner that the binding to the first vascular tissue is distinguishable from binding to a second vascular tissue. In other embodiments, a subject functionalized magnetic nanoparticle binds to a first bone tissue in such a manner that the binding to the first bone tissue is distinguishable from binding to a second bone tissue.

As one example, a subject functionalized magnetic nanoparticle will in some embodiments bind with at least about 20%, at least about 30%, at least about 40%, at least about 50%, at least about 70%, at least about 90%, at least about 2-fold, at least about 2.5-fold, at least about 3-fold, at least about 4-fold, at least about 5-fold, at least about 10-fold, or at least about 50-fold, or more, higher affinity to a first brain tissue than to a second brain tissue. As another example, a subject functionalized magnetic nanoparticle will in some embodiments bind with at least about 20%, at least about 30%, at least about 40%, at least about 50%, at least about 70%, at least about 90%, at least about 2-fold, at least about 2.5-fold, at least about 3-fold, at least about 4-fold, at least about 5-fold, at least about 10-fold, or at least about 50-fold, or more, higher affinity to a first vascular tissue than to a second vascular tissue. As one example, a subject functionalized magnetic nanoparticle will in some embodiments bind with at least about 20%, at least about 30%, at least about 40%, at least about 50%, at least about 70%, at least about 90%, at least about 2-fold, at least about 2.5-fold, at least about 3-fold, at least about 4-fold, at least about 5-fold, at least about 10-fold, or at least about 50-fold, or more, higher affinity to a first bone tissue than to a second bone tissue.

In some embodiments, the first brain tissue is a diseased brain tissue; and the second brain tissue is a normal, non-diseased brain tissue. In other embodiments, the first brain tissue is a normal (non-diseased) brain tissue; and the second issue is a diseased brain tissue. In other embodiments, the first brain tissue is a first, non-diseased brain tissue of a first tissue type; and the second brain tissue is a second, non-diseased brain tissue of a second tissue type. In other embodiments, the first brain tissue is a brain tissue before stimulation with an external or internal stimulus; and the second brain tissue is the same brain tissue after stimulation with an external or internal stimulus.

In some embodiments, the first vascular tissue is a diseased vascular tissue; and the second vascular tissue is a normal, non-diseased vascular tissue. In other embodiments, the first vascular tissue is a normal (non-diseased) vascular tissue; and the second tissue is a diseased vascular tissue. Diseased vascular tissue includes, e.g., vascular tissue that is inflamed, e.g., an inflammatory reaction occurs at or near the vascular tissue. In other embodiments, the first vascular tissue is a vascular tissue before compromise due to any external or internal cause; and the second vascular tissue is the same vascular tissue after compromise due to the same external or internal cause. Compromised vascular tissue is diseased or disturbed in any way such that it differs in at least one physiological parameter from normal vascular tissue. Inflamed vascular tissue is an example of compromised vascular tissue.

In some embodiments, the first bone tissue is a diseased bone tissue; and the second bone tissue is a normal, non-diseased bone tissue. In other embodiments, the first bone tissue is a normal (non-diseased) bone tissue; and the second tissue is a diseased bone tissue. Diseased bone tissue includes, e.g., bone tissue that is inflamed, e.g., an inflammatory reaction occurs at or near the bone tissue (e.g., bone destruction in inflammatory bone resorptive disorders such as osteoarthritis, rheumatoid arthritis, diabetes, and the like). In other embodiments, the first bone tissue is a bone tissue before compromise due to any external or internal cause; and the second vascular tissue is the same bone tissue after compromise due to the same external or internal cause. Compromised bone tissue is diseased or disturbed in any way such that it differs in at least one physiological parameter from normal bone tissue.

In some embodiments, a functional moiety is one that binds with greater affinity to a diseased brain tissue than to a non-diseased, normal brain tissue. In other embodiments, a functional moiety is one that binds with greater affinity to a normal brain tissue than to a diseased brain tissue. In some embodiments, a functional moiety is one that binds with greater affinity to a first, non-diseased brain tissue than to a second, non-diseased brain tissue. In other embodiments, a functional moiety is one that binds with greater affinity to a first brain tissue after stimulation with a external or internal stimulus than to the same brain tissue before stimulation with the external or internal stimulus.

In some embodiments, a functional moiety is one that binds with greater affinity to a diseased vascular tissue than to a non-diseased, normal vascular tissue. In other embodiments, a functional moiety is one that binds with greater affinity to a normal vascular tissue than to a diseased vascular tissue. In other embodiments, a functional moiety is one that binds with greater affinity to a first vascular tissue after compromise due to any external or internal cause than to the same vascular tissue before compromise due to the same external or internal cause.

In some embodiments, a functional moiety is one that binds with greater affinity to a diseased bone tissue than to a non-diseased, normal bone tissue. In other embodiments, a functional moiety is one that binds with greater affinity to a normal bone tissue than to a diseased bone tissue. In other embodiments, a functional moiety is one that binds with greater affinity to a first bone tissue after compromise due to any external or internal cause than to the same bone tissue before compromise due to the same external or internal cause.

Suitable functional groups include, but are not limited to, an antibody that binds specifically to an epitope(s) present in the brain, vascular, or bone tissue; a ligand that specifically binds to a receptor present on the plasma membrane of a cell of the brain, vascular, or bone tissue; a ligand that specifically binds to a receptor present in the cytoplasm of a cell of the brain, vascular, or bone tissue; a receptor or a receptor fragment that binds specifically to a component present in the brain tissue or on a cell present in the brain, vascular, or bone tissue; and the like. Exemplary, non-limiting functional groups include antibodies; neurotransmitters (e.g., GABA, glutamate, NMDA, opiates, opiate analogs, serotonin, 5HT1A, MPPA, and the like); cytokines (e.g., interleukins, such as IL-1 through IL-16, IFN-γ, IFN-α, IFN-β); receptor antagonists; and the like. Where the functional group is an antibody, suitable antibodies include whole antibodies (e.g., IgG), antibody fragments, such as Fv, F(ab′)₂ and Fab, chimeric antibodies, and the like.

Diseased tissue (e.g., brain tissue, vascular tissue, or bone tissue) can be imaged using a subject functionalized magnetic nanoparticle. Neurological diseases and disorders in which diseased brain tissue can be imaged include, but are not limited to, a brain tumor; multiple sclerosis (MS); Devic's disease (Devic's syndrome or Neuromyelitis Optica); human immunodeficiency virus (HIV) infection; Wallerian degradation; epilepsy; Parkinson's disease; Huntington's disease; amyotropic lateral sclerosis (ALD); Alzheimer's Disease (AD); Creutzfeld-Jacob Disease (CJD); drug dependency disorders, e.g., dependency on antidepressants, anxiolytic compounds, hallucinogenic compounds, or other psychoactive compounds; psychiatric disorders such as bipolar mood disorder, schizophrenia, and the like; etc.

Vascular diseases and disorders that can be imaged using a subject functionalized magnetic nanoparticle include, but are not limited to, inflammation and/or restenosis as a result of reanastomosis or transplant through vascular surgery, or inflammatory diseases of the peripheral or central vasculature resulting from diseases such as diabetes.

Bone diseases and changes that can be imaged using a subject functionalized magnetic nanoparticle include, but are not limited to, the bone changes that result from inflammatory response resulting from diabetes or chemicals or drugs, as well as neoplastic diseases originating from the bone tissues or metastasizing to the bone tissues.

In some embodiments, a functional moiety is one that binds with higher or lower affinity to epileptic tissues in the brain. Non-limiting examples of such functional moieties include: 1) glucose or a glucose derivative such as fludeoxyglucose, where the glucose is differentially taken up by epileptic tissues, compared to normal, non-epileptic tissues; 2) N-methyl-D-aspartate (NMDA), where the NMDA binds to receptors of epileptic tissue cells differentially, depending on an increase or a decrease in NMDA receptors on the cells; 3) α-methyl tryptophan, where α-methyl tryptophan is selectively taken up by epileptogenic tubers in intractable epilepsies in children with tuberous sclerosis; 4) cytokines such as tumor necrosis factor (TNF), and interleukins such as IL-1, IL-6, and IL-10, where an increased expression of IL-1 receptors, IL-6 receptors, or IL-10 receptors on epileptic tissue results in greater uptake of IL-1-conjugated magnetic nanoparticles or IL-6-conjugated magnetic nanoparticles by epileptic tissues; 5) γ-aminobutyric acid (GABA), where at the level of the GABA_{A} (GABA_{A}-α1-6, GABA_{A}-β1-3, GABA_{A}-γ2, GABA_{A}-δ, and GABA_{A}-ε receptor, neurodegeneration-induced loss in receptors is accompanied by markedly altered expression of receptor subunits in the dentate gyrus and other parts of the hippocampal formation, indicating altered physiology and pharmacology of GABA_{A} receptors; 6) an opiate or an opioid such as alfentanil, buprenorphine, carfentanil, codeine, dihydrocodeine, diprenorphine, etorphine, fentanyl, heroin, hydrocodone, hydromorphone, LAAM, levorphanol, meperidine, methadone, morphine, naloxone, naltrexone, β-hydroxy-3-methylfentanyl, oxycodone, oxymorphone, propoxyphene, remifentanil, sufentanil, tilidine, tramadol, and the like; 7) serotonin, e.g., 5-hydroxytryptamine-1A (5HTT1A), and other serotonin receptor agonists; 8) 3-methylphosphinicopropionic (MPPA); 9) benzodiazepines such as flumazenil, lorazepam, diazepam, alprazolam, brotizolam, chlordiazepoxide, clobazam, clonazepam, clorazepate, demoxepam, estazolam, flurazepam, halazepam, midazolam, nitrazepam, nordazepam, oxazepam, prazepam, quazepam, temazepam, and triazolam; 10) glutamate; and 11) acetylcholine and other acetylcholine receptor agonists.

In some embodiments, a functional moiety is one that binds differentially to a dopamine nerve terminal (e.g., D2 and D3 agonists and antagonists). Cocaine recognition sites are localized on the dopamine transporter, which itself is localized on dopamine nerve terminals. Drugs that bind to these sites therefore have potential uses which include: (i) imaging probes for neurodegenerative disorders; and (ii) imaging probes for dopamine transporter/cocaine binding sites. Suitable functional moieties that bind differentially to dopamine nerve terminals include N-haloallyl nortropane derivatives, such as Iodoaltropane. See, e.g., U.S. Pat. No. 5,853,696 for examples of such derivatives. Functionalized magnetic nanoparticles functionalized with an N-haloallyl nortropane derivative are useful for imaging neurodegenerative disorders associated with a loss of dopamine nerve terminals, such disorders including Parkinson's disease.

Suitable functional moieties include moieties that bind differentially to diseased brain tissue associated with Alzheimer's Disease (AD). Suitable functional moieties include agents that bind differentially to β-amyloid plaques; moieties that bind differentially to neurofibrillary tangles (NFT); moieties that bind to the CCR1 receptor (see, e.g., the compounds described in U.S. Pat. No. 6,676,926; and the like. Suitable functional moieties include, but are not limited to, compounds as described in U.S. Pat. No. 6,274,119; antibodies to β-amyloid protein; antibody to a component of a NFT; and the like.

Suitable functional moieties include moieties that bind differentially to a brain tumor, e.g., that bind differentially to an epitope expressed on the surface of a brain tumor cell. Brain tumor markers include markers for gliomas, astrocytomas, and the like. See, e.g., Lu et al. (2001) Proc. Natl. Acad. Sci. USA 98:10851; Boon et al. (2004) BMC Cancer 4(1):39.

Suitable functional moieties include moieties that bind differentially to brain tissue affected by multiple sclerosis; and moieties expressed on the surface of monocytes and/or CD4⁺ T cells that mediate the pathology of MS and that may be found in the vicinity of brain or other CNS tissue affected by MS.

Suitable functional moieties include moieties that bind differentially to brain tissue after exposure to an external or internal stimulus, compared to the same brain tissue before exposure to the external or internal stimulus. Such functional moieties include antibodies that bind to a receptor (e.g., a cell-surface receptor) that is up-regulated after exposure to an external or internal stimulus; a receptor ligand that binds to a receptor that is up-regulated after exposure to an external or internal stimulus; antibodies that bind to a receptor (e.g., a cell-surface receptor) that is down-regulated after exposure to al external or internal stimulus, a receptor ligand that binds to a receptor that is down-regulated after exposure to an external or internal stimulus, and the like. External and internal stimuli include, but are not limited to, electrical stimuli; drugs, e.g, psychoactive compounds, depressants (opioids, synthetic narcotics such as carfentanil, barbiturates, glutethimide, methyprylon, ethchlorvynol, methaqualone, alcohol); anxiolytics (flumazenil, diazepam, chlordiazepoxide, alprazolam, oxazepam, temazepam); stimulants (amphetamine, methamphetamine, cocaine); and hallucinogens (LSD, mescaline, peyote, marijuana; and the like; sound; heat; light; thoughts; stress; and the like.

### Compositions

The present invention further provides compositions, including pharmaceutical compositions, comprising a subject functionalized magnetic nanoparticle. Compositions comprising a subject functionalized magnetic nanoparticle will include one or more of the following: a salt; a buffer; a pH adjusting agent; a non-ionic detergent; a protease inhibitor; a nuclease inhibitor; and the like.

A pharmaceutical composition comprising a subject functionalized magnetic nanoparticle will comprise one or more pharmaceutically acceptable carriers. As used herein, “pharmaceutically acceptable carrier” includes any material which, when combined with an active ingredient of a composition, allows the ingredient to retain biological activity and without causing disruptive reactions with the subject's immune system or other physiological function. Examples include, but are not limited to, any of the standard pharmaceutical carriers such as a phosphate buffered saline solution, water, emulsions such as oil/water emulsion, and various types of wetting agents. Exemplary diluents for aerosol or parenteral administration are phosphate buffered saline or normal (0.9%) saline. Compositions comprising such carriers are formulated by well known conventional methods (see, for example, Remington's Pharmaceutical Sciences, Chapter 43, 14th Ed., Mack Publishing Col, Easton Pa. 18042, USA). Pharmaceutically acceptable excipients have been amply described in a variety of publications, including, for example, A. Gennaro (2000) “Remington: The Science and Practice of Pharmacy,” 20th edition, Lippincott, Williams, & Wilkins; Remington's Pharmaceutical Sciences, 14th Ed. or latest edition, Mack Publishing Col, Easton Pa. 18042, USA; Pharmaceutical Dosage Forms and Drug Delivery Systems (1999) H. C. Ansel et al., eds., 7^{th} ed., Lippincott, Williams, & Willdins; and Handbook of Pharmaceutical Excipients (2000) A. H. Kibbe et al., eds., 3^{rd} ed. Amer. Pharmaceutical Assoc.

A subject functionalized magnetic nanoparticle can be formulated into preparations for injection by dissolving, suspending or emulsifying in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Methods of Making a Functionalized Magnetic Nanoparticle that Cross the Blood-Brain Barrier

The present invention further provides methods of making a subject functionalized magnetic nanoparticle that crosses the blood-brain barrier (BBB). The methods generally involve attaching a functional group, either directly or via a linker, to a magnetic nanoparticle. In some embodiments, the magnetic nanoparticle is coated with a layer to which a functional group or a linker is attached, either covalently or non-covalently. The functionalized MNP is prepared for transfer across the BBB in any of several ways.

In some embodiments, the functionalized MNP further comprises an apolipoprotein (e.g., apoA, apoB, or apoE) attached to the functionalized MNP. The apolipoprotein provides for binding to endothelial cells of the BBB, and thus provides for transit of the functionalized MNP across the BBB.

In some embodiments, the functionalized MNP is further processed by attaching human serum albumin and/or apolipoprotein to the functionalized MNP. Human serum albumin (HSA) is attached, covalently or non-covalently (e.g., via ionic interactions) to the functionalized MNP via an acetyl group, via an amino group, via a poly(ethylene glycol) (PEG) linker, or via a thiol bond. Apolipoprotein, or a functional fragment thereof, is attached to the HSA, either covalently or non-covalently. See, e.g. Muller and Keck ((2004) J. Nanosci. Nanotechnol. 4:471); and Kreuter et al. ((2002) J. Drug Target. 10:317). Amino acid sequences of apolipoproteins are known in the art; for example, amino acid sequences of apoE polypeptides are found at e.g., GenBank Accession Nos. AAD02505; and AAB59397.

A functionalized MNP will in some embodiments be encapsulated in an HSA matrix, as described below.

In other embodiments, the functionalized MNP further comprises apolipoprotein attached to the functionalized MNP via polysorbate-80. In some embodiments, the functionalized MNP is further processed by attaching polysorbate-80 covalently or non-covalently to the functionalized MNP. In some embodiments, the polysorbate-80 is attached via an acetyl group, via an amino group, via a PEG linker, or via a thiol bond directly to the coating layer. Apolipoprotein is attached to the polysorbate-80, either covalently or non-covalently.

In other embodiments, the functionalized MNP is associated with (e.g. adsorbed onto, covalently linked to, non-covalently associated with) poly(butyl cyanoacrylate) (PBCA) particles, e.g., a functionalized NP is adsorbed onto the surface of a PBCA particle. In still other embodiments, the functionalized MNP comprises polysorbate-80 covalently or non-covalently attached to the functionalized MNP; and further comprises polybutyl cyanoacrylate).

Incorporation into Microorganisms

In some embodiments, a functionalized MNP or a non-functionalized MNP is incorporated into a microorganism, e.g., a bacterium or a virus. A microorganism that comprises a functionalized or non-functionalized M-NP is useful for visualization (imaging) of the location and/or movement of such microorganism in vivo.

### MRI-Visible Drug Deliver System

The present invention provides a magnetic resonance imaging (MRI)-visible drug delivery system; and method of synthesizing same. A subject MRI-visible drug delivery system comprises a functionalized MNP, as described above, where the functionalized MNP comprises at least one drug (e.g., a therapeutic agent). A subject NMR-visible drug delivery system is useful in some embodiments for determining the distribution of a drug in the body. A subject MRI-visible drug delivery system is useful in other embodiments for tissue-specific drug delivery. For example, where a subject functionalized MNP comprises both a tissue-specific binding moiety and a therapeutic agent, the functionalized MNP is a tissue-specific drug delivery system. In some embodiments, a subject drug delivery system is adapted for crossing the BBB, e.g., the drug delivery system comprises one or more elements that provide for crossing the BBB.

As one non-limiting example, a first functional group provides for binding to an epileptic tissue in the brain; and a second functional group is a therapeutic agent that treats epilepsy. A therapeutic agent that treats epilepsy includes, but is not limited to, dilantin (phenyloin sulfate); tegretol (carbamazepine); epilim (sodium valproate); zarontin (ethosuximide); rivertril (clonazepam); frisium (clobazepam); and the like.

### Utility

The present invention further provides various applications in which a subject functionalized magnetic nanoparticle finds utility, including research applications, diagnostic applications, and treatment applications.

### Diagnostic Methods

The present invention provides diagnostic methods for identifying or detecting a specific brain tissue. The methods generally involve administering to an individual a subject functionalized magnetic nanoparticle; and imaging an area of the brain to which the functionalized magnetic nanoparticle is bound. Typically, a liquid pharmaceutical composition comprising a subject functionalized magnetic nanoparticle is injected into the individual (e.g., intravenous injection); and the functionalized magnetic nanoparticle is detected by an imaging technique. In many embodiments, the imaging is by magnetic resonance imaging. The methods of the invention thus permit imaging of a particular brain tissue in a living subject. The methods of the invention permit detection of diseased tissue in the brain, and also provide a way for physicians to monitor the progress of patients undergoing treatment for the disease.

A subject diagnostic method is useful for diagnosing the presence of a neurological disease and/or for monitoring the response of an individual to a treatment for a neurological disease or disorders including, but not limited to, a brain tumor; multiple sclerosis (MS); epilepsy; Parkinson's disease; Huntington's disease; amyotropic lateral sclerosis (ALD); Devic's disease; Alzheimer's Disease (AD); Creutzfeld-Jacob Disease (CJD); Cortical Dysplasia; Rasmussen's encephalitis; drug dependency disorders, e.g., dependency on antidepressants, anxiolytic compounds, hallucinogenic compounds, or other psychoactive compounds; psychiatric disorders such as bipolar mood disorder, schizophrenia, and the like; etc.

The present invention provides methods of identifying a vascular tissue at risk of restenosis. The method generally involves administering to an individual subject functionalized magnetic nanoparticle; and imaging a vascular tissue to which the functionalized magnetic nanoparticle is bound. In some embodiments, the vascular tissue is imaged using a subject functionalized magnetic nanoparticle that is functionalized with a functional group that differentially binds to inflamed vascular tissue, compared with normal vascular tissue. In some embodiments, the functional group is an inflammatory cytokine, or a moiety (e.g., an antibody or antigen-binding fragment thereof) that binds an inflammatory cytokine. Suitable cytokines include IL-1 through IL-16, and TNF-α.

In addition, immunologically active cells loaded with unconjugated MNPs bind to the surface of vascular tissues and can be used in a subject method for identifying vascular tissue, e.g., diseased vascular tissue. Suitable cells include monocytes, T cells (e.g., CD4⁺ T cells), and the like.

The present invention also provides methods for detecting diseased bone tissue in an individual. The method generally involves administering to an individual a subject functionalized magnetic nanoparticle; and imaging a bone tissue to which the functionalized magnetic nanoparticle is bound. In some embodiments, the bone tissue is imaged using a subject functionalized magnetic nanoparticle that is functionalized with a functional group that differentially binds to diseased bone tissue. In some embodiments, the functional group is an inflammatory cytokine, or a moiety (e.g., an antibody or antigen-binding fragment thereof) that binds an inflammatory cytokine. Suitable cytokines include IL-1 through IL-16, and TNF-α.

In addition, immunologically active cells loaded with unconjugated MNPs bind to the surface of bone tissues and can be used in a subject method for identifying bone tissue, e.g., diseased bone tissue. Suitable cells include monocytes, T cells (e.g., CD4⁺ T cells), and the like.

The present invention further provides methods for detecting diseased vascular or bone tissue, e.g., vascular tissue affected by inflammation or bone tissue affected by inflammation, in an individual. The method generally involves administering to an individual a magnetic nanoparticle that is not functionalized, such that the magnetic nanoparticle binds the inflamed vascular tissue or inflamed bone tissue; and imaging the diseased vascular or bone tissue using an imaging technique such as MRI.

### Research Applications

The present invention provides research applications using a subject functionalized magnetic nanoparticle. A subject functionalized magnetic nanoparticle is injected into a subject, and the functionalized magnetic nanoparticle is detected by imaging. Research applications include assaying the effect of a given test agent on a particular disease. Research applications Her include testing the effect of various external and internal stimuli on normal and diseased brain tissue. Research applications further include testing the effect of various compromising causes (external or internal) on normal and diseased vascular or bone tissue.

### Screening Methods

Research applications include screening methods for assaying the effect of a given test agent on a particular disease. Thus, in some embodiments, the present invention provides methods of identifying a candidate therapeutic agent for a neurological disorder, the method involving administering a test agent to an experimental (non-human) animal model of a neurological disorder (e.g., an experimental animal model of multiple sclerosis, Alzheimer's Disease, brain tumor, epilepsy, etc.); and determining the effect, if any, of the test agent on a neurological feature associated with the neurological disorder. Determining the effect of the test agent is carried out by administering to the non-human animal model a composition comprising a subject functionalized magnetic nanoparticle, where the functionalized magnetic nanoparticle exhibits differential binding to a diseased brain tissue affected by or associated with the neurological disorder; and detecting the functionalized magnetic nanoparticle in the brain of the animal. Detection is typically by magnetic resonance imaging.

Neurological features associated with a particular neurological disorder include, e.g., the size of an epileptic lesion (for epilepsy); the size of a brain area affected by multiple sclerosis (for multiple sclerosis); the size and/or number of β-amyloid plaques, the size and/or number of NFT (for Alzheimer's Disease); the size of a brain tumor (for brain tumors); and the like. Animal models of various neurological disorders are known in the art. For example, for multiple sclerosis (MS), the experimental autoimmune encephalitis (EAE; also referred to in the literature as the experimental allergic encephalitis) model is a rodent model of MS. Various mouse models of AD are available; see, e.g., Buttini et al. (1999) J Neurosci. 19(12):4867-80.

The terms “candidate agent,” “test agent,” “agent,” “substance,” and “compound” are used interchangeably herein. Candidate agents encompass numerous chemical classes, typically synthetic, semi-synthetic, or naturally-occurring inorganic or organic molecules. Candidate agents include those found in large libraries of synthetic or natural compounds. For example, synthetic compound libraries are commercially available from Maybridge Chemical Co. (Trevillet, Cornwall, UK), ComGenex (South San Francisco, Calif.), and MicroSource (New Milford, Conn.). A rare chemical library is available from Aldrich (Milwaukee, Wis.). Alternatively, libraries of natural compounds in the form of bacterial, fungal, plant and animal extracts are available from Pan Labs (Bothell, Wash.) or are readily producible.

Candidate agents may be small organic or inorganic compounds having a molecular weight of more than 50 and less than about 2,500 daltons. Candidate agents may comprise functional groups necessary for structural interaction with proteins, particularly hydrogen bonding, and may include at least an amine, carbonyl, hydroxyl or carboxyl group, and may contain at least two of the functional chemical groups. The candidate agents may comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

A screening assay typically includes controls, where a suitable control includes an experimental animal having the neurological disorder, and not treated with the test agent.

A test agent of interest is one that reduces a neurological feature of the disorder by at least about 10%, at least about 20%, at least about 25%, at least about 30%, at least about 35%, at least about 40%, at least about 45%, at least about 50%, at least about 55%, at least about 60%, at least about 65%, at least about 70%, at least about 80%, at least about 90%, or more, when compared to a control in the absence of the test agent.

The present invention is also useful for identifying particular mediator(s) of immune reactions responsible for restenosis of vascular anastomosis as performed in peripheral and central vascular surgery in a variety of disorders that require this surgical intervention. The present invention is also useful for identifying specific predictors of vascular restenosis secondary to vascular anastomosis by providing a method for MRI imaging the particular anastomosis that are prone to restenosis, through their reaction with specifically tagged magnetonanoparticles.

The present invention is also useful for identifying, through MRI, particular mediator(s) of immune reaction responsible for inflammation and injury of bone as occur due to diabetes. The present invention is also useful for identifying specific predictors of bone inflammation and injury due to diabetes by providing a method for MR imaging the bone tissues that are prone to inflammation and injury, through their reaction with specifically tagged magnetonanoparticles (MNP).

### Treatment Applications

The present invention provides methods of treating a disease, disorder, or condition, the method generally involving administering to an individual in need thereof an effective amount of a subject functionalized MNP. In some of these embodiments, the subject functionalized MNP comprises a therapeutic agent (“drug”) and a functional moiety that provides for tissue-specific (e.g., diseased tissue-specific) targeting.

In some embodiments, a pharmaceutical composition comprising a subject functionalized MNP is administered to an individual in need thereof, where the subject functionalized MNP comprises a therapeutic agent. In some embodiments, a subject pharmaceutical composition comprising a subject functionalized MNP is administered to an individual in need thereof, where the subject functionalized MNP comprises a therapeutic agent, where the route of administration is parenteral, e.g., intravenous, intramuscular, subcutaneous, intratumoral, intracranial, peritumoral, etc.

An effective amount of a subject functionalized MNP is an amount that is sufficient to at least ameliorate the symptoms of a disease, disorder, or condition. In some embodiments, an effective amount of a subject functionalized MNP is an amount that is effective to reduce the severity and/or incidence of at least one symptom of a disease or disorder by at least about 10%, at least about 20%, at least about 25%, at least about 30%, at least about 40%, at least about 50%, at least about 60%, at least about 70%, at least about 80%, at least about 90%, or more, compared to the severity and/or incidence of the symptom in an individual not treated with the functionalized MNP.

An effective amount of a subject functionalized will vary, depending on various factors including, e.g., the nature of the disease, disorder, or condition; the severity or extent of the disease, disorder, or condition; the age or other physical characteristics of the individual; and the like. Effective amounts include, e.g., from about 10² to about 10¹⁸ functionalized MNP, e.g., from about 10² to about 10³ functionalized MNP, from about 10³ to about 10⁴ functionalized MNP, from about 10⁴ to about 10⁵ functionalized MNP, from about 10⁵ to about 10⁶ functionalized MNP, from about 10⁶ to about 10⁷ functionalized MNP, from about 10⁷ to about 10⁸ functionalized MNP, from about 10⁸ to about 10⁹ functionalized MNP, from about 10⁹ functionalized MNP to about 10¹⁰ functionalized MNP, from about 10¹⁰ functionalized MNP to about 10¹² functionalized MNP, from about 10¹² functionalized MNP to about 10¹⁴ functionalized MNP, from about 10¹⁴ functionalized MNP to about 10¹⁶ functionalized MNP, or from about 10¹⁶ functionalized MNP to about 10¹⁸ functionalized MNP.

Unit doses of functionalized MNP will comprise from about from about 10² to about 10¹⁸ functionalized MNP, e.g., from about 10² to about 10³ functionalized MNP, from about 10³ to about 10⁴ functionalized MNP, from about 10⁴ to about 10⁵ functionalized MNP, from about 10⁵ to about 10⁶ functionalized MNP, from about 10⁶ to about 10⁷ functionalized MNP, from about 10⁷ to about 10⁸ functionalized MNP, from about 10⁸ to about 10⁹ functionalized MNP, from about 10⁹ functionalized MNP to about 10¹⁰ functionalized MNP, from about 10¹⁰ functionalized MNP to about 10¹² functionalized MNP, from about 10¹² functionalized MNP to about 10¹⁴ functionalized MNP, from about 10¹⁴ functionalized MNP to about 10¹⁶ functionalized MNP, or from about 10¹⁶ functionalized MNP to about 10¹⁸ functionalized MNP.

In some embodiments, multiple doses of a functionalized MNP will be administered. For example, a unit dose of a functionalized MNP will be administered is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid). In some embodiments, a functionalized MNP is administered at any suitable frequency, and over a period of time ranging from about one day to about one week, from about two weeks to about tour weeks, from about one month to about two months, from about two months to about four months, from about four months to about si months, from about six months to about eight months, from about eight months to about 1 year, from about 1 year to about 2 years, or from about 2 years to about 4 years, or more.

Individuals in need of treatment include individuals having any of a variety of disorders, particularly brain or CNS disorders, e.g., individuals having MS, epilepsy, Parkinsons' disease, etc. Individuals in need of treatment include individuals having vascular disorders, e.g., vascular disorders that arise as a result of diabetes; individuals having or at risk of having restenosis; and the like.

The present invention provides methods of treating a disease, disorder, or condition, the method generally involving administering to an individual in need thereof an effective amount of a subject functionalized MNP, where the subject functionalized MNP comprises a functional moiety that provides for tissue-specific targeting of the MNP. In some embodiments, e.g., where the disease is epilepsy, where the functionalized MNP comprises a functional moiety for targeting the MNP to epileptic tissues. The functionalized MNP are administered to an individual having epilepsy; the functionalized MNP bind to epileptic tissues; and the tissues are heated by exposure to electromagnetic radiation, to ablate the diseased tissue. Electromagnetic radiation includes, e.g., radiation of from about 100 kiloHertz (kHz) to about 1000 kHz.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Preparation of Functionalized Magnetic Nanoparticles

### Nanoparticle Preparation

200 mg of human serum albumin (USA) are dissolved in 2.0 ml water containing magnetic nanoparticles (MNP; e.g., magnetite particles). The pH of the solution is raised to 8.4 under constant stirring by dropwise addition of 0.01 M and 0.1 M solution of NaOH. Under constant stirring desolvatation of the 10% HSA solution is performed by dropwise addition of 8.0 ml ethanol. After addition of ethanol, 235 μl of an 8% glutaraldehyde solution are added. After 24 h, the resulting nanoparticles are purified by threefold centrifugation (16.100 g, 8 min) and redispersion in water. Redispersion is performed in an ultrasonication bath. HSA-MNP synthesized using this method have an average diameter of about 60 nm to about 990 nm, depending on the pH of the preparation and addition of non-conjugated or conjugated MNP. AMT-MNP nanoparticles have an average diameter of approximately 20 Dill, and a size range of from about 10 nm to about 40 nm.

### Preparation of Neutravidin™-Modified NP

### NeutrAvidin™ Binding to Nanoparticles

Purified nanoparticles are activated using the crosslinker NHS-PEG3400-Mal (Nektar, Huntsville, USA; where “NHS” is N-hydroxysuccinimide, and “Mal” is maleimide, and “PEG3400” is poly(ethylene glycol) having an average molecular weight of 3400 daltons) in order to achieve a sulfhydryl-reactive particle system. A volume of 500 μl crosslinker solution (NHS-PEG3400-Mal, 60 mg/ml in PBS-buffer pH 8.0) is added to 2.0 ml nanoparticle (NP) dispersion (20 mg/ml in PBS-buffer pH 8.0). The mixture is incubated under shaking for 1 h at room temperature. Afterwards, the activated nanoparticles are purified by centrifugation and redispersion as described above.

Subsequently, NeutrAvidin™ is conjugated to the activated HSA-NP by heterobifunctional crosslinking as described. NeutrAvidin™ is non-glycosylated avidin. An aliquot (10.0 mg) NeutrAvidin™ is dissolved in 1.0 ml TEA-buffer (pH 8.0) and 1.2 mg 2-iminothiolane (Traut's reagent) in 1.0 ml TEA-buffer (pH 8.0) is added. After 12 h incubation at room temperature, the thiolated protein is purified by size exclusion chromatography (D-Salt™ Desalting Column). For the conjugation 1 ml thiolated and purified NeutrAvidin™ solution are added to 1 ml sulfhydryl-reactive human serum albumin (HSA) nanoparticles. The mixture is incubated under shaking for 12 h at room temperature. The non-reacted thiolated NeutrAvidin™ is removed by NP centrifugation and redispersion in water. The supernatants of the centrifugation steps are assayed spectrophotometrically at 280 nm to determine uncoupled NeutrAvidin™.

### ApoE Surface Modification of Neutravidin™-Modified Nanoparticles

### ApoE Biotinylation

To enable the attachment of apoE to NeutrAvidin™-modified nanoparticles, ApoE is biotinylated according to a standard protein modification protocol with PFP-Biotin (Pierce, Rockford, USA. PFP-biotin is pentafluorophenyl ester of biotin. ApoE is dissolved in PBS pH 7.0 at a concentration of 167 μg/ml. The biotinylated protein is separated from low molecular weight compounds by a dextran desalting column. The efficiency of the biotinylation process is determined by western blot as described below.

Binding of Biotinylated apoE to NeutrAvidin™-Modified Nanoparticles

The drug-loaded NeutrAvidin™-modified nanoparticles are redispersed in water to a particle concentration of 20 mg/ml. Subsequently, 167 μg biotinylated apoE (biotin-apoE) are added resulting in a final concentration of 10 mg/ml NP and 80 μg/ml apoE. After 12 h incubation the NP supernatant is analyzed for unbound apoE by immunoblotting as described below.

### Drug Loading of Nanoparticles

Approximately 20 mg of the purified NeutrAvidin™-modified HSA-MNP are incubated with 6.6 mg drug in an ethanol/water solution. After an incubation period of 2 h, the unbound drug is removed by centrifugation and redispersion.

### Covalent Binding of ApoE to Nanoparticles Via a Peg Crosslinker

HSA nanoparticles are activated using the crosslinker NHS-PEG3400-Mal in order to achieve a sulfhydryl-reactive particle system as described above. Subsequently, apoE is conjugated to the activated HSA nanoparticles by heterobifunctional crosslinking. Aliquots (500 μg) of different apoE-derivatives (apoE3, apoE2 Arg142Cys, ApoE Sendai) are dissolved in 1.0 ml TEA-buffer (pH 8.0) and 2-Iminothiolane (Traut's reagent) is added in a 50-fold molar excess concentration. After a 12 h incubation period at room temperature, the thiolated protein is purified by size exclusion chromatography (D-Salt™ column). For the conjugation 500 μg thiolated and purified apoE is added to 25 mg sulfhydryl-reactive HSA nanoparticles. The mixture is incubated under shaking for 12 h at room temperature. The unreacted thiolated apoE is removed by centrifugation and redispersion of the particles in ethanol/water (2.6% ethanol v/v).

Approximately 20 mg of the purified apoE-modified HSA nanoparticles is incubated with 6.6 mg drug in an ethanol/water solution. After an incubation time of 2 h, the unbound drug is removed by centrifugation. The dug-loaded apoE-PEG nanoparticles are redispersed in water.

### Preparation of Polysorbate 80-Coated HSA Nanoparticles

Nanoparticles (NP) without ApoE but coated with polysorbate 80 are prepared by adsorption of the drug to NeutrAvidin™-modified nanoparticles as described above. Then, the drug-loaded nanoparticles are incubated with polysorbate 80 (1% n/v) solution for 30 min and used.

### Preparation of Tissue-Specific Ligand Modification of HSA-MNP

Tissue-specific ligands such as α-methyl tryptophan (AMT), neurotransmitters, etc., are coupled to free amino or carboxyl groups in the HSA, or are coupled via polycarbon linkers (e.g., PEG), or through thiol bonds or other attachment moieties.

### Preparation of Poly(Butyl Cyanoacrylate)-MNP

0.1 g stabilizer (either dextran 70,000 or Pluronic F68) was added to 10 ml 0.001 M HCl under constant stirring. Two solutions were prepared: 1) One solution contained 0.1 g Dextran 70,000 (Sigma-Aldrich) in 10 ml 0.001 M HCl; and 2) a second solution contained 0.1 g Pluronic F68 (Sigma, Inc.) in 10 ml 0.001 M HCl. The following four preparations were prepared: 1) Non-functionalized MM) were added to the Pluronic F68 solution; 2) non-functionalized MM) were added to the Dextran solution; 3) functionalized MM) (AMT-MNP) were added to the Pluronic F68 solution; and 4) functionalized MM) (AMT-MNP) were added to the Dextran solution. Under stirring at 500 rpm, 100 μg cyanoacrylate monomers (Sicomet, Sichel-Werke, GmbH) was added to each preparation slowly just below the surface of the fluid.

Each solution was kept for 2-2.5 hours, with stirring. After this period, each solution was neutralized by addition of 990 μl 0.1 N NaOH. Finally, each solution was filtered.

Drugs are added to the solution between 1 minute and 30 minutes after the start of stirring.

Surfactant is not added to preparations having Pluronic F68 as stabilizer. 1 mg Polysorbate 80 is added to 100 ml of the particle solution when dextran is used as stabilizer. The functionalized MNP prepared as described above have a diameter in a range of from about 80 nm to about 350 nm; and have a zeta potential of between ⁻10 mV and ⁻50 mV, e.g., about ⁻30 mV.

### Synthesis of AMT-Functionalized MNP

Dextran-coated maghemite (γ-Fe₂O₃) MNPs functionalized with α-methyl tryptophan (AMT) were prepared as follows.

The structure of AMT is depicted below

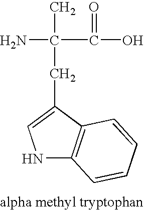

The chemical structure of the dextran polymer is generally:

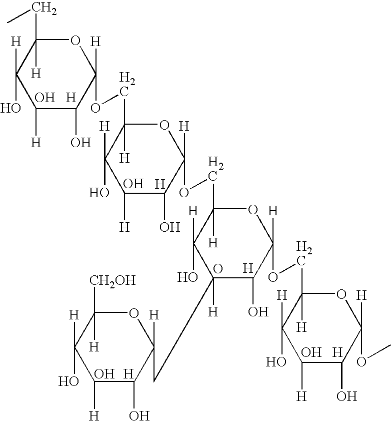

The reaction is depicted schematically as follows:

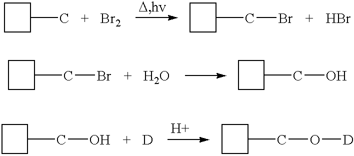

where

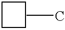

represents AMT; and “D” represents dextran.

AMT coupled to an MNP surface via the α-methylene group is depicted below.

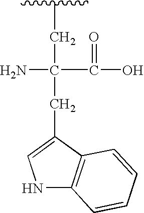

Modified AMT is depicted below:

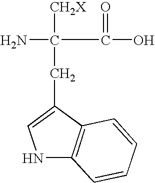

where X is Hal, SH, NH₂, or other group that provides for attachment.

### TEM Images of Functionalized MNP

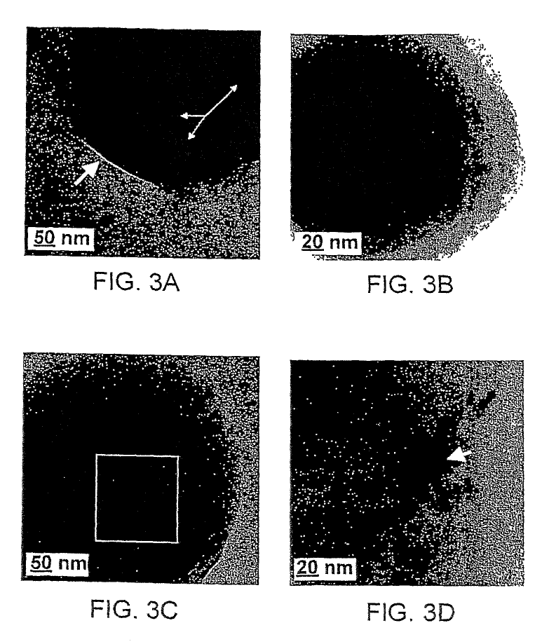

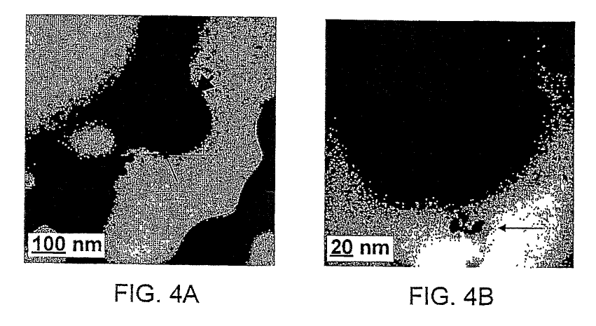

FIGS. 3A-3D depict transmission electron microscope (TEM) images of AMT-MNP within an HSA matrix, prepared as described above. FIG. 3A depicts an HSA-MNP particle; HSA (arrowhead) and AMT-MNP (arrows) are shown. FIG. 3B depicts AMT-MNP particles in HSA matrix. FIG. 3C depicts another distribution of MNP; and FIG. 3D depicts a magnification of the area set out in the black box in FIG. 3C, showing the presence of magnetic particles (TEM-dense regions, arrowhead) in the core of the MNPs. FIGS. 4A and 4B depict TEM micrographs of PBCA-MNP, prepared as described above. FIG. 4A depicts PBCA particles (arrowhead) and AMT-MNP (arrow) adsorbed to the surface of the PBCA particle. FIG. 4B depicts a magnification of the area set out in the black box in FIG. 4A. The magnification depicted in FIG. 4B shows the adsorption of AMT-MNP (arrow) to the surface of the PBCA particle.

### Example 2

### In Vivo Characterization of Functionalized MNP

Non-functionalized MNP and AMT-conjugated MNP were administered to a kainic acid (KA) model of epilepsy. The data demonstrated that AMT-MNP display affinity for epileptic tissues.

Two Lewis rats (90 days old) were injected in the right hippocampus with 1 μl KA solution. The rats developed status epilepticus immediately post-injection with KA. Status epilepticus stopped approximately 48 hours post-injection. On day 3 post KA injection, baseline MRI were obtained, using T2 sequences (TR=6000 ms; TE=50 ms; slice thickness=1.5 mm; interstices distance=0.25 mm). After the baseline MRI, the first rat was injected (i.v.) with AMT-MNP (300 μmol/kg) and the second rat was injected (i.v.) with non-functionalized MNP (300 μmol/kg). MRI were repeated 6 hours after each rat was injected with the MNP.

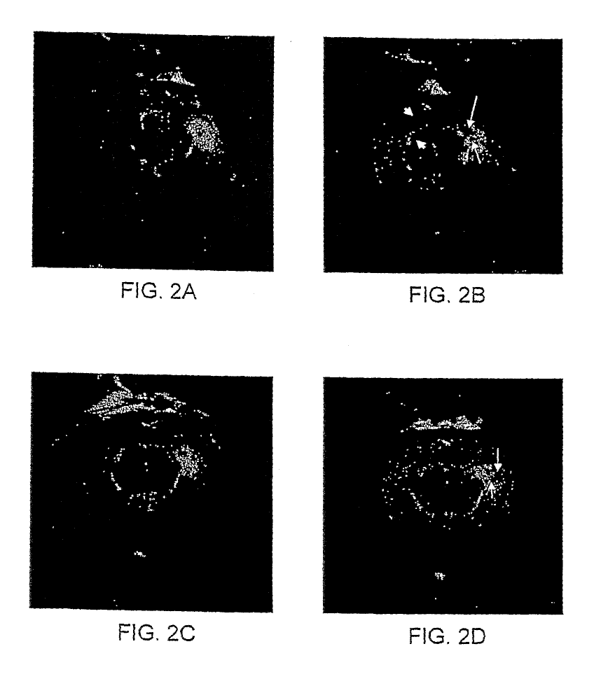

FIG. 2A shows baseline MRI of the first rat; FIG. 2B shows the areas of (negative) enhancement in the CA1 (upper arrowhead) and dentate gyrus (lower arrowhead) contralateral to the site of KA injection in this AMT-MNP-treated rat. These changes were absent from the identically prepared rat treated with non-functionalized MNP (FIGS. 2C and 2D). The signal changes in the contralateral CA1 and dentate gyrus in the AMT-MNP-treated rat are consistent with tissue changes associated with acute epilepsy. These data suggest the affinity of the AMT-MNP for epileptic tissues.

FIG. 2B also shows the areas of (negative) enhancement (white arrows) in the right hippocampus ipsilateral to the site of AMT-MNP injection. FIG. 2C shows the baseline M of the non-functionalized MNP-treated rat. FIG. 2D shows the areas of (negative) enhancement in the right hippocampus ipsilateral to the site of KA injection (white arrows). The signal changes in the right hippocampus of both animals are consistent with the expected inflammatory response at the sites of KA injection. The signal enhancement is thought to be due to the presence of magnetically tagged particles in either macrophages which enter the brain parenchyma through transcytosis or incorporation of the nanoparticles by resident glial cells; these cells are thought to be mediators of inflammatory response in the brain.

The signal changes in the contralateral CA1 and dentate gyrus in the AMT-MNP-treated rat are consistent with tissue changes associated with acute epilepsy and are not likely related to inflammatory response. The areas of enhancement in the hippocampus are due to acute inflammatory response to KA injection in both rats, while the signal changes in the CA1 and dentate gyrus are attributable to acute epileptic discharges and affinity of the AMT-conjugated particles for these epileptic tissues.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A pharmaceutical composition comprising:
a) a functionalized magnetic nanoparticle (MNP) comprising a functional group that has differential affinity for a tissue in the brain, wherein said functionalized magnetic nanoparticle is capable, when introduced into the bloodstream of a mammalian subject, of crossing the blood-brain barrier of said subject and binding specifically to the brain tissue; and
b) a pharmaceutically acceptable carrier.

2. The composition of claim 1, wherein the tissue is a diseased tissue.

3. The composition of claim 2, wherein the diseased tissue is selected from a brain tumor, an epileptic lesion, a plaque associated with Alzheimer's Disease, a tissue affected by multiple sclerosis, a tissue affected by Huntington's Disease, a tissue affected by Parkinson's Disease, and a tissue affected by amyotropic lateral sclerosis.

4. The composition of claim 1, wherein the tissue is one that is exposed to an external or internal stimulus.

5. The composition of claim 1, wherein said functional group is an antibody that specifically binds an epitope present in the brain tissue.

6. The composition of claim 1, wherein said functional group is a ligand that binds specifically to a receptor present on or in a cell present in the brain tissue.

7. The composition of claim 1, wherein the functionalized MNP further comprises a therapeutic agent.

8. The composition of claim 1, wherein the functionalized MNP is encapsulated in an albumin matrix.

9. The composition of claim 1, where the functionalized MNP comprises an apolipoprotein.

10. The composition of claim 1, wherein the functionalized MNP comprises poly(butyl cyanoacrylate) (PBCA).

11. The composition of claim 10, wherein the functionalized MNP is attached to the surface of a PBCA particle.

12. The composition of claim 1, wherein the functionalized MNP comprises a surfactant.

13. The composition of claim 12, wherein the surfactant is selected from polyoxyethylene sorbitan monooleate, polyoxyethylene sorbitan monopalmitate, polyoxyethylene sorbitan monostearate, and polyoxyethylene sorbitan monolaurate.

14. The composition of claim 12, wherein the surfactant is a block copolymer of polyethylene oxide and polypropylene oxide.

15. The composition of claim 12, wherein the functionalized MNP comprises a poloxamine.

16. A pharmaceutical composition comprising:
a) a functionalized magnetic nanoparticle comprising a functional group that has differential affinity for an inflamed vascular tissue that is at risk of restenosis, wherein said functionalized magnetic nanoparticle is capable, when injected into the bloodstream of a mammalian subject, of binding specifically to the inflamed vascular tissue; and
b) a pharmaceutically acceptable carrier.

17. A pharmaceutical composition comprising:
a) a functionalized magnetic nanoparticle comprising a functional group that has differential affinity for a diseased bone tissue, and wherein said functionalized magnetic nanoparticle is capable, when injected into the bloodstream of a mammalian subject, of binding specifically to the diseased bone tissue; and
b) a pharmaceutically acceptable carrier.

18. The composition of claim 17, wherein the bone tissue is inflamed as a result of diabetes, injury, or other compromising factors causing an inflammatory response in the bone tissue.

19. A pharmaceutical composition comprising:
a) a magnetic nanoparticle that is derivatized with a functional group that has differential affinity for epileptic tissue in the brain, and wherein said magnetic nanoparticle is capable, when injected into the bloodstream of a mammalian subject, of crossing the blood-brain barrier of said subject and binding specifically to epileptic tissue in the brain; and
b) a pharmaceutically acceptable carrier.

20. The pharmaceutical composition of claim 19, wherein the functional group is glucose.

21. The pharmaceutical composition of claim 19, wherein the functional group is N-methyl-D-aspartate.

22. The pharmaceutical composition of claim 19, wherein the functional group is α-methyl tryptophan.

23. The pharmaceutical composition of claim 19, wherein the functional group is a cytokine.

24. The pharmaceutical composition of claim 19, wherein the functional group is γ-amino butyric acid.

25. The pharmaceutical composition of claim 19, wherein the functional group is an opiate or an opioid compound.

26. A method of diagnosing a brain disorder, the method comprising:
a) administering to a mammalian subject a composition comprising a functionalized magnetic nanoparticle, wherein said functionalized magnetic nanoparticle comprises a functional group that has differential affinity for a tissue in the brain that is affected by the brain disorder, and wherein said functionalized magnetic nanoparticle is capable, when injected into the bloodstream of the mammalian subject, of crossing the blood-brain barrier of said subject; and
b) detecting the presence of the functionalized magnetic nanoparticle in the brain.

27. The method of claim 26, wherein the brain disorder is selected from a brain tumor, epilepsy, Alzheimer's Disease, multiple sclerosis, Huntington's Disease, Parkinson's Disease, amyotropic lateral sclerosis, a drug addition, and a psychiatric disorder.

28. The method of claim 26, wherein the composition is administered by intravenous injection.

29. The method of claim 26, wherein said detecting is by magnetic resonance imaging.

30. A method of detecting a vascular tissue that is at risk of restenosis, the method comprising:
a) administering to a mammalian subject a composition comprising a functionalized magnetic nanoparticle, wherein said functionalized magnetic nanoparticle comprises a functional group that has differential affinity for an inflamed vascular tissue, and wherein said functionalized magnetic nanoparticle is capable, when injected into the bloodstream of the mammalian subject, of binding specifically to the inflamed vascular tissue; and
b) detecting the presence of the functionalized magnetic nanoparticle in the vascular tissue.

31. A method of detecting a diseased bone tissue in a mammalian subject, the method comprising:
a) administering to a mammalian subject a composition comprising a functionalized magnetic nanoparticle, wherein said functionalized magnetic nanoparticle comprises a functional group that has differential affinity for diseased bone tissue, and wherein said functionalized magnetic nanoparticle is capable, when injected into the bloodstream of the mammalian subject, of binding specifically to the diseased bone tissue; and
b) detecting the presence of the functionalized magnetic nanoparticle in the bone tissue.

32. A method of identifying an agent that treats a brain disorder, the method comprising:
administering a test agent to a non-human animal model of a brain disorder; and
determining the effect, if any, of the test agent on a neurological feature of the brain disorder, wherein said determining is carried out by: i) administering to the non-human animal model a composition comprising a functionalized magnetic nanoparticle, wherein the functionalized magnetic nanoparticle exhibits differential binding to a diseased brain tissue affected by or associated with the neurological disorder; and ii) detecting the functionalized magnetic nanoparticle in the brain of the animal.

33. The method of claim 32, wherein said detecting is by magnetic resonance imaging.

34. A method of treating a disorder in an individual, the method comprising:
administering to an individual in need thereof an effective amount of a composition of claim 1.

35. The method of claim 34, wherein the functionalized MNP further comprises a therapeutic agent that treats the disorder.
